(19) United States
(12) Patent Application Publication
(10) Pub. No.: US 2014/0180265 A1
(43) Pub. Date: Jun. 26, 2014

(54) OPHTHALMOLOGICAL LASER METHOD AND APPARATUS

(71) Applicant: Cheng-Hao Huang, Orlando, FL (US)

(72) Inventor: Cheng-Hao Huang, Orlando, FL (US)

(21) Appl. No.: 14/190,526

(22) Filed: Feb. 26, 2014

Related U.S. Application Data
(63) Continuation-in-part of application No. 13/373,591, filed on Nov. 22, 2011.

(51) Int. Cl. A61F 9/008

(52) U.S. Cl. CPC A61F 9/00814
USPC 606/5

(57) ABSTRACT

The present invention relates to a femtosecond laser ophthalmological apparatus and method that creates a flap on the cornea for LASIK refractive surgery or for other applications that require removal of corneal and lens tissue at specific locations such as in corneal transplants, stromal tunnels, corneal lenticular extraction and cataract surgery. The femtosecond laser is transferred to a hand piece module via a rotating mirror arm module. In the hand piece, the femtosecond laser beam is scanned into overlapping circles of laser pulses which are then moved in an overlapping trajectory on a patient's eye to ablate the eye tissue in a predetermined pattern.

### BACKGROUND OF THE INVENTION

This patent application is a continuation-in-part application of my pending U.S. patent application Ser. No. 13/373,591, filed Nov. 22, 2011 for OPHTHALMOLOGICAL LASER METHOD & APPARATUS.

The present invention relates to a femtosecond laser ophthalmological apparatus and method that creates a flap on the cornea for LASIK refractive surgery or for other applications that require removal of corneal and lens tissue at specific locations such as in corneal transplants, stromal tunnels, corneal lenticular extraction and cataract surgery.

The use of an excimer laser to modify the shape of the cornea is called Laser Vision Correction (LVC). Currently the most popular method is called LASIK (Laser-Assisted in-situ Keratomileusis) and accounts for approximately 85% of all LVC preformed. Traditionally, during LASIK, the surgeon uses an instrument called a mechanical microkeratome (physical blade) to create a flap on the cornea. However, over the last few years, femtosecond laser has increasingly been used to create a LASIK flap using a series of hundreds of thousands of small laser pulses to create a cleavage plane (“cut”) in the cornea.

Femtosecond laser created corneal flaps can offer greater safety, reproducibility, predictability and flexibility over mechanical microkeratome. Furthermore, complications such as buttonhole flaps (in very steep corneas), free caps (in very flat corneas) and irregular flaps that are associated with the mechanical microkeratome are rare with the femtosecond laser. Finally, femtosecond laser systems offer a wide range of other optical-related applications to include corneal transplants, stromal tunnels, corneal lenticular extraction and cataract surgery.

However, several limitations are currently associated with a femtosecond laser system: The overall size of current femtosecond laser systems are much larger than mechanical microkeratome systems. Concurrently, with the exception to Ziemer Ophthalmic AG's Femto LDV systems, current femtosecond laser systems require the patient's eye to be aligned to a fixed laser beam delivery point. These two factors negatively impacts patient and surgeon comfort during surgery. Whereas the corneal flap creation by mechanical microkeratome and subsequent corneal reshaping by an excimer laser system can be done without moving the patient, the size of femtosecond laser systems and fixed delivery require patients to be transferred from one location to another. It is not uncommon for patients to have to move to a separate room to receive corneal reshaping. The femtosecond laser created flaps also increases the surgery time (decreased workflow) as there is often a necessary wait time after laser flap creation (for cavitation gas bubbles to diffuse) before the patient can be moved. This is also a significant reason why most Ophthalmology clinics in the world still employ mechanical microkeratome for more efficient workflow.

U.S. Pat. No. 7,621,637 by Rathjen describes an ophthalmological apparatus that Ziemer Ophthalmic AG's Femto LDV series currently utilizes, and it addresses the size, flexibility of delivery and surgery time (small laser spot size for smaller cavitation gas bubbles). However, there are several disadvantages to this current design. First, the scanning pattern impinges the scan lens at varying incident angles and requires an extra optical system to continuously maintain beam focal points on the same focal plane. A method of repositioning these points during the scan so that they are all on the focal plane is a primary concern of another Rathjen U.S. Pat. No. 7,597,444. Second, to avoid pattern distortion on the eye created by the translation motor, the laser pulse line scanning pattern has to be precisely aligned to be perpendicular with the trajectory of the translation motor after passing through the delivery arm. Rathjen compensates with a rotation element to maintain the perpendicular trajectory. This creates a more complex, less reliable and potentially more expensive apparatus. A third disadvantage of this apparatus is that the width of the scanned subarea may vary if the trajectory is not linear. This limits the trajectories available to the target. With a constraint on the trajectories available, other possible optical-related applications are limited (especially when a 3D curvature is required inside the cornea or on the lens tissue). Finally, this patent uses a mirror-lens relay optical arm to deliver the laser beam from the main cabinet into the hand piece. Placing lenses in an optical arm amplifies alignment errors and creates a more complex module.

In the present invention, an ophthalmological apparatus utilizes a femtosecond laser beam that travels through a rotating mirror set module as opposed to a mirror-lens relay optical arm. Using only mirrors simplifies the optical system's design and operation. The rotating mirror set module is attached to the main cabinet and a hand piece where the laser beam is deflected by a two dimensional XY scanner device into an overlapping, generally circular scanning pattern of laser pulses. The pattern impinges the zoom-able scan focusing lens in the hand piece at a constant distance from the center axis and results in focal points in the same focal plane and eliminates the need for compensating optics as required by the prior art. Furthermore, the generally circular scanning pattern is automatically aligned perpendicularly with the trajectory of a XY piezo positioning stage since the overlapping circular scanning pattern is not sensitive to any rotational factors induced by the XY piezo positioning stage. This removes the need for the rotation element described in the prior art. The resultant ablation patterns are of a consistent line width that is not sensitive to the direction of the trajectory. This provides flexibility for different trajectories such as line patterns, concentric circle patterns and spiral patterns that is not easily attainable in the prior art.

### SUMMARY OF THE INVENTION

A ophthalmological apparatus utilizing a method of ablating eye tissue includes generating a pulsed laser beam from a femtosecond laser and directing the generated laser beam through a beam expander, a computer controlled electronically activated shutter and a rotating mirror set module so the laser beam enters the hand piece module at normal incidence to its entrance plane. The laser beam is applied to a two dimensional XY scanner in the hand piece module to generate an overlapping, generally circular scanning pattern of laser pulses (henceforth referred to as “circular pattern”). The circular pattern is applied co-axially to a zoom-able scan focusing lens supported by an XY piezo positioning stage in the hand piece module, and it focuses the circular pattern onto the patient's eye. The circular pattern is moved by the XY piezo positioning stage in an overlapping trajectory onto a patient's eye to ablate a predetermined pattern of eye tissue in the patient's eye. Such overlapping trajectories include concentric circles, parallel lines, spiral patterns and other regular and irregular topologies.

### BRIEF DESCRIPTION OF THE DRAWINGS

The accompanying drawings, which are included to provide further understanding of the invention, constitute a part of the specification and illustrate the invention together with the description to explain the principles of the invention.

In the drawings:

FIG. 1 is a diagrammatic view of the exterior of the critical modules of a femtosecond laser ophthalmological surgery apparatus in accordance with the present invention;

FIG. 2 is a block diagram of a femtosecond laser ophthalmologic surgery apparatus in accordance with the present invention;

FIG. 3 is a diagrammatic view of the hand piece module of FIGS. 1 and 2 showing the change in femtosecond laser characteristic as it exits the mirror set module and applied to the two dimensional XY scanner to become an overlapping, generally circular scanning pattern of laser pulses and then focused by the zoom-able scan focusing lens;

FIG. 4 shows an overlapping, generally circular scanning pattern of laser pulses impinging a zoom-able scan focusing lens at equidistance (R) from the center of the lens; and

FIG. 5 shows an overlapping, generally circular scanning pattern of laser pulses impinging a zoom-able scan focusing lens at equidistance (R) from the center of the lens that focuses the circular pattern on the same focal plane.

### DETAILED DESCRIPTION OF EXEMPLARY EMBODIMENT

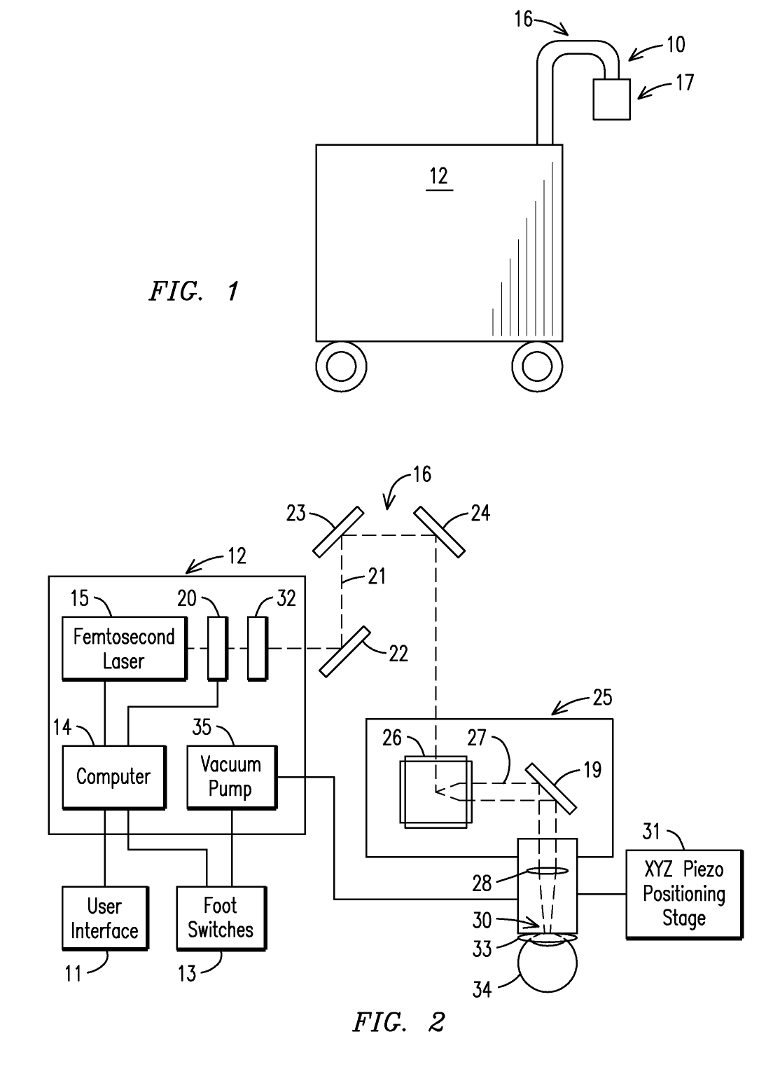

A laser ophthalmological surgery apparatus 10 in accordance with the present invention in FIGS. 1 and 2 of the drawings includes a user interface 11 connected to a main cabinet 12. A foot switch 13 and the user interface 11 are connected to a computer 14 inside the main cabinet 12. Within the main cabinet 12, laser pulses are generated with a femtosecond laser 15 that is guided through an attached rotating mirror set module 16. The ophthalmological apparatus 10 has a main cabinet 12 and a hand piece module 17 connected to either end of a rotating mirror set module 16. A laser beam expander 20 is positioned in the main cabinet 12 to enlarge the laser beam spot size before it is directed through the rotating mirror set module 16. The laser beam 21 passes through the rotating mirror set module 16 having mirrors 22, 23 and 24, even though it will be understood that more than three mirrors can be used without departing from the spirit and scope of the invention. The laser beam then passes into the hand piece module 25 at a normal incidence to the two dimensional XY scanner 26. The two dimensional XY scanner 26 deflects the laser beam 21 to create an overlapping, generally circular scanning pattern of laser pulses 27 (henceforth referred to as “circular pattern”). Inside the hand piece 25, a mirror 19 directs the beam into a zoom-able scan focusing lens 28 which is used to reduce the laser beam spot size 30 and the diameter of the circular pattern. The lens 28 is supported by an XY piezo positioning stage 31 in the hand piece module 25 that is used to move the circular pattern according to a predetermined trajectory onto the patient's eye.

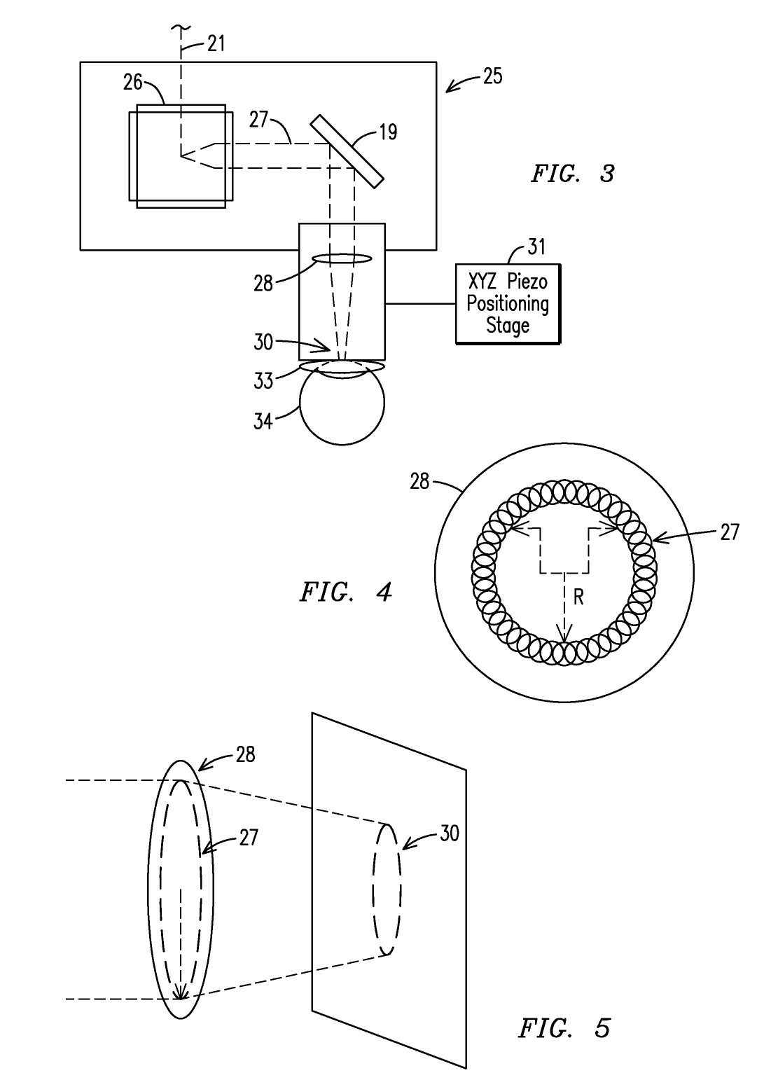

Following the light path in greater detail, the light pulse generator or femtosecond laser 15 has a pulse width of less than 1000 femtosecond and a pulse repetition rate greater than 10 KHz. The laser beam spot size is enlarged by a beam expander 20 and is then blocked by the shutter 32 until the foot switch 13 is depressed. While the foot switch 13 is depressed, the beam is allowed to continue through the rotating mirror set module 16 and into the hand piece module 25 as shown in FIG. 2. Inside the hand piece module 25, as seen in FIGS. 2 and 3, the laser beam 21 is deflected by the two dimensional XY scanner 26 into an overlapping, generally circular scanning pattern of laser pulses 27 as seen in FIG. 4 and also referred herein as “circular pattern”. The circular pattern may have a repetition rate up to 2000 KHz.

Inside the hand piece, as seen in FIG. 3, a compact, low F-number (high numeric aperture) zoom-able scan focusing lens 28 is mounted on an XY piezo positioning stage 31 to reduce the spot size of the laser beam to less than 3 microns and the size of the overlapping circular scanning pattern 27 to a diameter less than 800 microns. The zoom-able scan focusing lens 28 has a smaller size and higher focusing capability than the F-theta lens that is currently most commonly used in femtosecond ophthalmic systems. The higher focusing capability generates a smaller laser beam spot size 30 that reduces the required energy based on the same energy density. Reduced energy levels form smaller cavitation bubbles and are absorbed faster by the surrounding tissue. This also means the acoustic wave impact caused by the photo disruption is reduced. Furthermore, the smaller lens size can be incorporated into a smaller hand piece module that makes it easier to integrate with existing UV laser ophthalmic apparatus for LASIK.

The hand piece 25 is attached to the eye 34 with a clear disposable suction ring 33. The disposable suction ring 33 is placed on the eye 34 and secured using the low pressure created and maintained by a vacuum pump 35. The hand piece 25 is connected to the disposable suction ring 33 by a translation track. The laser beam 21 is reflected by mirror 19 and passes through the disposable suction ring 33 into the cornea of the eye 34. The suction ring 33 is placed on the eye 34 and aligned using a microscope. Once aligned, the hand piece 25 is manually pushed into the proper position as dictated by the suction ring 33. Since the suction ring 33 is completely clear when viewed under the microscope, alignment is easily performed.

In FIG. 4 the overlapping generally circular scanning pattern 27 of the laser beam are shown equidistance from the center of the scan focusing lens 28 circular pattern focused to the same focal plane. The lens forms a reduced spot size pattern 30 as seen in FIG. 5 which shows a plan view of the overlapping generally circular pattern of pulses 27 below the lens 28.

It should be clear at this time that a femtosecond laser eye surgery method and apparatus has been described which highlights the advantages of delivering a laser beam remotely using a rotating mirror set module 16 (as opposed to mirrors and lenses) into a hand piece module 25 and using an overlapping, generally circular scanning pattern 27 of laser pulses for surgery ablation pattern. However, it should be clear that the present invention is not to be considered as limited to the forms shown which are to be considered illustrative rather than restrictive.

I claim:

1. A method of ablating eye tissue comprising the steps of:
selecting an eye surgery apparatus having a femtosecond laser connected to a hand piece module with a rotating mirror arm having a plurality of mirrors therein;
generating a laser beam from the femtosecond laser source;
directing the generated laser beam through the rotating mirror arm into said hand piece module at normal incidence to its entrance plane;
applying the generated laser beam to a laser scanner in said hand piece to generate an overlapping, generally circular scanning pattern of laser pulses;
selecting an XYZ piezo positioning stage supported by a zoom-able scan focusing lens;
applying the overlapping, generally circular scanning pattern of laser pulses generated by said laser scanner onto the zoom-able scan focusing lens;
focusing the overlapping, generally circular scanning pattern of laser pulses onto a patient's eye; and
scanning the overlapping, generally circular scanning pattern of laser pulses onto the patient's eye to ablate a predetermined pattern of eye tissue on the patient's eye;
whereby a patient's eye can be ablated in a predetermined trajectory with a controlled focus of a femtosecond laser with overlapping circles of laser pulses.

2. The method of ablating eye tissue in accordance with claim 1 which includes scanning the femtosecond laser generated laser beam with a two dimensional XY scanner to generate the overlapping, generally circular scanning pattern of laser pulses.

3. The method of ablating eye tissue in accordance with claim 2 which includes scanning the overlapping, generally circular scanning pattern of laser pulses in a trajectory of generally concentric overlapping circular laser pulses.

4. The method of ablating eye tissue in accordance with claim 3 which includes scanning the overlapping, generally circular laser scanning pattern of laser pulses in a trajectory of regular or irregular topologies of overlapping circular laser pulses.

5. The method of ablating eye tissue in accordance with claim 1 including mounting said selected XYZ piezo positioning stage supported zoom-able scan focusing lens in an adjustable hand piece module.

6. The method of ablating eye tissue in accordance with claim 1 which includes mounting said femtosecond laser source in a main cabinet and said laser scanner in said hand piece module connected to the main cabinet by said rotating mirror set module.

7. The method of ablating eye tissue in accordance with claim 6 including the step of selecting a beam expander and applying said femtosecond laser beam to the beam expander in the main cabinet to enlarge the femtosecond laser beam spot size.

8. The method of ablating eye tissue in accordance with claim 6 which includes selecting a hand piece having a suction ring for attaching said hand piece to a patient's eye.

9. The method of ablating eye tissue in accordance with claim 6 in which the femtosecond laser beam is blocked by a shutter until activated by a foot switch.

10. An ophthalmological apparatus comprising:
a main cabinet and a hand piece module connected to said main cabinet by a rotating mirror arm module;
a femtosecond laser source positioned in said main cabinet and having a laser beam output of laser pulses;
a laser beam expander positioned to enlarge said femtosecond laser beam laser pulses;
a laser scanner positioned in said hand piece module for scanning said laser beam into an overlapping circular scanning pattern of laser pulses;
a XYZ piezo positioning stage located in said hand piece; and
a focusing lens mounted to said XYZ piezo positioning stage and positioned for receiving the overlapping circular scanning pattern of laser pulses from said laser scanner, centered on the center axis of said focusing lens, said focusing lens positioned to focus said overlapping circular scanning pattern of laser pulses onto a patient's eye while scanning said overlapping circular scanning pattern of laser pulses in a predetermined overlapping trajectory with said XYZ piezo positioning stage to ablate a predetermined pattern of tissue on the patient's eye;
whereby a patient's eye can have a predetermined area thereon ablated by said overlapping circles of laser pulses.

11. An ophthalmological apparatus in accordance with claim 10 in which said laser scanner is a two dimensional XY scanner.

12. An ophthalmological apparatus in accordance with claim 11 in which said rotating mirror set module has a plurality of mirrors therein.

13. An ophthalmological apparatus in accordance with claim 12 in which said rotating mirror set module has three mirrors therein.

14. An ophthalmological apparatus in accordance with claim 10 including a manually activated shutter mounted between said femtosecond laser source and said rotating mirror set module.

15. An ophthalmological apparatus in accordance with claim 10 in which said hand piece module has a suction ring for attaching said hand piece to a patient's eye.
